(19) United States
(12) Patent Application Publication
(10) Pub. No.: US 2026/0014390 A1
(43) Pub. Date: Jan. 15, 2026

(54) APPARATUS FOR STIMULATING AN AUDITORY NERVE

(71) Applicant: FRAUNHOFER-GESELLSCHAFT ZUR FÖRDERUNG DER ANGEWANDTEN FORSCHUNG E.V., München (DE)

(72) Inventors: Uwe VOGEL, Dresden (DE); Philipp WARTENBERG, Dresden (DE); Bernd RICHTER, Dresden (DE); Dirk SCHLEBUSCH, Dresden (DE); Johannes ZELTNER, Dresden (DE); Simone LENK, Dresden (DE); Karsten FEHSE, Dresden (DE); Andreas FRITSCHER, Dresden (DE); Stephan BRENNER, Dresden (DE)

(21) Appl. No.: 19/329,881

(22) Filed: Sep. 16, 2025

Related U.S. Application Data
(63) Continuation of application No. PCT/EP2024/056955, filed on Mar. 15, 2024.

(30) Foreign Application Priority Data
Mar. 16, 2023 (DE) 102023202410.1

(51) Int. Cl. A61N 5/06; H10K 59/80

(52) U.S. Cl. CPC A61N 5/0622; A61N 5/0601; A61N 2005/0652; A61N 2005/0653; A61N 2005/0664; H10K 59/879

(57) ABSTRACT

The application relates to an apparatus for stimulating an auditory nerve with a (CMOS) chip; a plurality of optical elements for stimulating the auditory nerve; and at least one driver circuit for controlling the plurality of optical elements. The plurality of optical elements and the at least one driver circuit are integrated in the (CMOS) chip.

### CROSS-REFERENCE TO RELATED APPLICATIONS

This application is a continuation of copending International Application No. PCT/EP2024/056955, file Mar. 15, 2024, which is incorporated herein by reference in its entirety, and additionally claims priority from German Application No. 102023202410.1, filed Mar. 16, 2023, which is also incorporated herein by reference in its entirety.

### TECHNICAL FIELD

The present application relates to an apparatus for stimulating an auditory nerve, in particular a fully integrable cochlear implant.

### BACKGROUND OF THE INVENTION

The basic mode of operation of a multi-channel cochlear implant has long been known [1]. It helps people whose inner ear has damage, but who still have an intact auditory nerve. Conventional solutions bridge the damaged inner ear with an external microphone and speech processor on the one hand and an implanted electrode arrangement on the auditory nerve. The auditory nerve itself is directly stimulated in this arrangement by electrical impulses. The number of electrode stimulation points here defines the differently perceptible frequencies and thus contributes significantly to the actually perceived hearing impression.

Newer approaches follow a promising optical approach instead of stimulation with electrodes [2], in which the stimulation takes place by μLEDs and optogenetic manipulation of the auditory nerve. In this arrangement, micro-LED chips are built up on a flexible substrate carrier and controlled externally. In this respect, the setup corresponds to the conventional method of external electronics and implanted “stimulation electrode”, wherein the production is very complex [3]: Thus, the required substrate flexibility is begun by a complex method with a polyimide layer on an Si wafer. Further steps of spin-coating, RIE, sputter, lift-off, electroplating (for an integrated temperature sensor) follow before the μLED itself is contacted. The achieved integration density is very low: 2-3 metal wiring levels, individual μLEDs and a temperature sensor are achieved. This does not correspond to much more than the known technology with conventional electrode arrangements. There is also the challenge of the connection between implanted electronics and external electronics (and an associated ergonomic/aesthetic impairment).

### SUMMARY

According to an embodiment, an apparatus for stimulating an auditory nerve may have: a chip; a plurality of optical elements for stimulating the auditory nerve; and at least one driver circuit for controlling the plurality of optical elements; wherein the plurality of optical elements and the at least one driver circuit are integrated monolithically in the chip; and wherein the chip is implemented as a CMOS chip.

The inventive apparatus for stimulating an auditory nerve is based on the finding that optical elements, for example, can be positioned at a very small distance from one another by CMOS technology and, in addition, a driver circuit for controlling the optical elements can also be integrated in the same substrate as the optical elements. As a result of the joint integration of the driver circuit and the optical elements in a single chip, improvements in terms of switching frequency, control power and dynamic losses are achieved when compared to previous hearing aids, in particular since the driver circuit can be arranged close to or even directly at or even below the optical elements. By means of a (CMOS) process, the individual components can be integrated in a single chip and linked to one another. As a result, a complete system is provided which does not require any SCT (setup and connection technology) whatsoever and thus allows simple production of the inventive apparatus. Furthermore, it has been achieved that the previously only externally implementable electronics, such as, for example, the driver circuit, could be integrated directly together with the stimulation unit, for example an array of a plurality of optical elements, in a common chip, as a result of which the stimulation unit can be implanted together with the electronics. Consequently, the inventive apparatus for stimulating the auditory nerve can be fully implanted and thus overcomes the challenge of the connection between implanted stimulation unit and external electronics. A further advantage of the present invention is that a large number of optical elements can be integrated in the (CMOS) chip and, consequently, a good frequency resolution can be achieved when stimulating the auditory nerve. The invention is based, among other things, on the finding that a CMOS chip meets biocompatibility requirements and, in particular, can be configured in such a flexible manner that the inventive (CMOS) chip can be bent in a spiral shape and can accordingly be introduced into a cochlea and implanted. Other production technologies apart from CMOS would also be conceivable. The novel method is furthermore characterized in that, according to embodiments, all functional elements (light generation, wiring, active circuit elements, temperature sensor) are integrated monolithically on the wafer level. Consequently, standard processes from semiconductor production can be used. The flexibility is achieved by a special method in which the wafer stack is extremely thinned from the front side and the rear side and the substrate becomes flexible by this.

A corresponding embodiment relates to an apparatus, e.g. an optoelectronic hearing aid, for stimulating an auditory nerve with a (CMOS) chip, a plurality of optical elements for stimulating the auditory nerve, and at least one driver circuit for controlling the plurality of optical elements. The plurality of optical elements and the at least one driver circuit are integrated in the (CMOS) chip.

The chip can be implemented e.g. as a semiconductor chip, like as a CMOS chip. According to an embodiment, the plurality of optical elements and the at least one driver circuit are integrated monolithically in the semiconductor chip. The term “monolithic” here relates e.g. to the fact that the plurality of optical elements and the at least one driver circuit are produced on a single semiconductor substrate, generally silicon, e.g. without bonding steps or SCT steps. Semiconductor-based light transmitters, e.g. light emitting diodes (LEDs or μLEDs) or laser diodes, can be used as optical (active) elements in semiconductor chips. By means of processes from semiconductor production, like from CMOS technology, the optical elements are integrated into the semiconductor substrate together with the at least one driver circuit. If the chip is implemented as a COMS chip, then the optical elements are produced by the same method as other CMOS devices, for example. The production process comprises creating the structures and doping regions within the semiconductor substrate in order to form the optical elements. This process generally comprises steps like oxidation, layer deposition, photolithography, etching, doping, annealing (temperature treatment) and/or metallization, similar to those used when producing CMOS transistors. Apart from using CMOS technology, the optical elements can also be integrated monolithically in the chip together with the at least one driver circuit using III-V semiconductor materials, like GaN (gallium nitride). The monolithic configuration is based on the finding that a chip configured in such a way has a high resistance in the liquid environment within the cochlea and thus contributes to the longevity of the apparatus. Furthermore, the production method is simplified essentially by the monolithic configuration.

A hybrid configuration is also conceivable in which the components, i.e. the optical elements and the driver circuit, were produced from different semiconductor materials or were produced by means of different technologies. Thus, for example, the driver circuit can be integrated in the chip by means of CMOS processes and the optical elements can be integrated in the chip using III-V semiconductor materials, or vice versa. According to an embodiment, the at least one driver circuit can be integrated in the chip by means of CMOS, using thin-film transistors (TFTs) or using III-V semiconductor material or SiC material, and the plurality of optical elements can be integrated in the chip by means of CMOS, using III-V semiconductor material or using OLED.

According to an embodiment, the chip can be implemented as a layer stack, wherein the at least one driver circuit is arranged in a first layer stack region and the plurality of optical elements are arranged in a second layer stack region, wherein the first layer stack region and the second layer stack region are arranged vertically one above the other. Wiring or connection planes are optionally arranged between the first layer stack region and the second layer stack region. The wiring or connection planes can be realized by means of CMOS technology. The plurality of optical elements are formed in the form of OLEDs in the second layer stack region, for example, by organic materials being applied on a main surface region of a layer of the layer stack, for example using techniques such as thermal vacuum evaporation or organic vapor deposition (OVPD). These organic layers generally comprise an emitting layer, a hole transport layer, an electron transport layer and other functional layers. As described in connection with the figures, an embodiment of a chip with OLED can be realized by embedding a plurality of pixel electrodes in a layer with organic material. The pixel electrodes are connected to the at least one driver circuit, for example. Each pixel electrode is optionally connected to its own driver circuit. An embodiment relates to a chip with a CMOS driver circuit in connection with optical elements in the form of OLEDs. A particular advantage of OLED on CMOS is that the OLED layers can be applied monolithically on the CMOS substrate, generally by evaporation. The OLED layers can optionally be encapsulated with a very thin layer. The thin encapsulation layer is applied directly on the OLED layers, for example by atomic layer deposition (ALD), chemical vapor deposition (CVD) or sputtering. When integrating the plurality of optical elements and the at least one driver circuit in the chip, no bonding or SCT processes are used. This makes the apparatus particularly robust for the liquid environment within the cochlea and allows very efficient production of the apparatus.

According to an embodiment, the apparatus has a plurality of the driver circuits, which are associated bijectively to the plurality of optical elements. Each of the plurality of the driver circuits is configured to control one of the plurality of optical elements. The apparatus thus has a driver circuit for controlling the respective optical element for each of the plurality of optical elements. The driver circuits are integrated in the (CMOS) chip. This allows flexible and individual driving of each individual optical element of the apparatus. It is particularly advantageous if the respective driver circuit is arranged in the immediate vicinity of or below the respective optical element which is controlled by the driver circuit, i.e. if the respective driver circuit is arranged in the immediate vicinity of the optical element associated with the driver circuit. Advantageous dynamics, in particular a high duty cycle, can be achieved by this.

According to an embodiment, one or more components from the group comprising a speech processor, a wireless interface, a microphone and an energy supply for providing energy obtained from cell energy, chemical energy, thermal energy or movement energy are also integrated in the (CMOS) chip. The speech processor corresponds, for example, to an electronic unit for acoustic data preprocessing, e.g. for converting an acoustic signal into an electrical signal. The wireless interface is configured, for example, to transmit or transfer data and/or energy between an external unit and the apparatus, e.g. for power supply/energy supply and/or data transmission. The wireless interface is configured, for example, to transmit the data and/or the energy via an electrical field, via a magnetic field, by light or mechanically. The microphone is configured, for example, to receive acoustic signals. The speech processor is configured, for example, to receive and process the acoustic signals received by the microphone, i.e. to convert them into electrical signals. The energy supply is configured, for example, to obtain the energy from cell energy, chemical energy or motion energy. A unit attached externally to the ear can be reduced in size by the additional integration of one or more of these components. Depending on which of the components are integrated, an external unit may even be completely dispensed with under certain circumstances, as a result of which the system can be fully implanted. External interference effects, ergonomic impairments and/or aesthetic impairments can be reduced by integrating one or more of the components.

According to an embodiment, the (CMOS) chip has a first portion, which is configured to be introduced into the cochlea. Furthermore, the (CMOS) chip has a second portion, which adjoins/is adjacent to the first portion, for example, and which is configured to be arranged outside the cochlea. The (CMOS) chip has, for example, a common substrate for the first portion and the second portion, i.e. the (CMOS) chip is implemented monolithically. The plurality of optical elements and the at least one driver circuit are integrated in the first portion of the (CMOS) chip and the one or more components mentioned above, i.e. the speech processor, the wireless interface, the microphone and/or the energy supply, are integrated in the second portion of the (CMOS) chip. The first portion thus forms a stimulation unit, which can be introduced into the scala tympani of the cochlea, for example. The optical units are configured, for example, to emit light and thus specifically stimulate the auditory nerve. The auditory nerve is optogenetically manipulated, for example, in order to be able to be stimulated by light. The second portion of the (CMOS) chip is not intended to be introduced into the cochlea, for example. This special division has the advantage, in particular, that the components mentioned above are not located in the liquid contained in the scala vestibuli and scala tympani. High robustness and longevity of the apparatus are achieved by this. Furthermore, it has been recognized that, for example, in particular the microphone can receive acoustic signals with high quality outside the cochlea, which is why it is advantageous to arrange it in the second portion. A further advantage of this arrangement is that the first portion of the (CMOS) chip, which is to be introduced into the approximately pea-sized cochlea, has small dimensions, i.e. a very small diameter, since the at least one driver circuit and the optical elements can be realized in the (CMOS) chip to be very small.

According to an embodiment, the apparatus has an RF antenna, which is integrated in the first portion of the (CMOS) chip. Data can be transmitted between the apparatus and an external unit by means of the RF antenna. The RF antenna is used, for example, for communication with the external unit or further external devices.

According to an embodiment, the (CMOS) chip has a thickness, i.e. an extension perpendicular to a plane in which the plurality of optical elements are arranged, of at most 100 μm, 90 μm, 80 μm, 70 μm or down to 20 μm. High flexibility of the apparatus is achieved by this so that it can follow the shape of the cochlea. Damage within the cochlea when introducing the apparatus can thus be reduced, as a result of which a high hearing quality can be achieved by the apparatus after implanting the same.

According to an embodiment, the apparatus has at least one beam-shaping element, for example a lens, such as, for example, a convex lens or a converging lens. The (CMOS) chip has, for example, a first main surface, which is faced, for example, by the plurality of optical elements. An optical element of the plurality of optical elements is configured to couple out light via the first main surface within an emission region, and the at least one beam-shaping element is arranged or fixed in the emission region on the first main surface and is configured, for example, to shape, bundle and/or focus the light of the optical element. In a plan view, for example, the beam-shaping element and the optical element are arranged to be completely overlapping. The beam-shaping element and the optical element are aligned, for example, along the same axis, wherein the axis represents, for example, an axis of symmetry of the beam-shaping element and of the optical element. The apparatus optionally has a beam-shaping element for each of the plurality of optical elements, i.e., the apparatus has a plurality of beam-shaping elements. One, for example optically active, diaphragm, like a light-impermeable element, is arranged, for example, between two adjacent beam-shaping elements of the plurality of beam-shaping elements. The auditory nerve can be stimulated very specifically by means of the beam-shaping element. Consequently, the distance between the optical elements of the plurality of optical elements can be reduced since the light is provided in a very focused manner and the emitted light of two adjacent optical elements thus does not overlap or hardly overlaps. The optical elements can thus be positioned at a small distance from one another and individual groups of nerve cells of the auditory nerve can nevertheless be stimulated, as a result of which a high frequency resolution can be achieved in the auditory perception of the user.

According to an embodiment, the (CMOS) chip has a first main surface and a second main surface opposite the first main surface. The (CMOS) chip has a transparent or semi-transparent region for coupling out light of the plurality of optical elements via the first main surface and via the second main surface, i.e. the (CMOS) chip is implemented to be transparent or semi-transparent in one or more regions. The material of the COMS chip is translucent/transparent in this region or these regions, for example.

According to an embodiment, the optical elements of the plurality of optical elements are configured to emit light in two opposite directions. When the apparatus is inserted into the cochlea, the apparatus winds around the auditory nerve. However, the apparatus can twist in this case, as a result of which the optical elements in a certain region may not face the auditory nerve under certain circumstances and, consequently, a reduced stimulation quality is achieved in this region. This can be counteracted by the inventive emission of light in two opposite directions. It is achieved by this that the apparatus achieves a high stimulation quality even when the (CMOS) chip is twisted within the cochlea.

According to an embodiment, the (CMOS) chip is configured as a layer stack and the plurality of optical elements is arranged in a first layer of the layer stack and the at least one driver circuit is arranged in a second layer of the layer stack. Further layers can be arranged between the first layer and the second layer.

According to an alternative embodiment, the (CMOS) chip is configured as a layer stack and the plurality of optical elements have a first set of optical elements and a second set of optical elements. The first set of optical elements is arranged in a first layer of the layer stack and the second set of optical elements is arranged in a second layer of the layer stack. The optical elements of the first set of optical elements face a first main surface of the (CMOS) chip and the optical elements of the second set of optical elements face a second main surface of the (CMOS) chip, wherein the second main surface corresponds to a surface of the (CMOS) chip opposite the first main surface. The optical elements of the first set thus emit light in the opposite direction to the optical elements of the second set. Similarly, as already explained above for optical elements emitting on two sides, it can also be achieved in this embodiment that the apparatus achieves a high stimulation quality even when the (CMOS) chip is twisted within the cochlea since the stimulation light is coupled out on two opposite sides of the (CMOS) chip by the special arrangement of the optical elements. It is particularly advantageous if the optical elements of the first set are aligned with the optical elements of the second set such that, for example, one axis, for example an axis of symmetry, of an optical element of the first set coincides with an axis, for example an axis of symmetry, of an optical element of the second set. An optical element of the first set and an optical element of the second set are thus opposite each other within the (CMOS) chip, for example.

According to an embodiment, the at least one driver circuit is arranged in a third layer of the layer stack, wherein the third layer is arranged between the first layer and the second layer. For example, two opposite optical elements optionally always share a driver circuit arranged therebetween. Consequently, the driver circuit is arranged very close to the optical elements to be controlled, as a result of which advantageous dynamics, in particular a high duty cycle, can be achieved.

According to an embodiment, in a plan view, the at least one driver circuit overlaps at least partially with at least one of the plurality of optical elements. Alternatively, in the plan view, the at least one driver circuit is arranged between two optical elements of the plurality of optical elements arranged adjacently within a plane or layer. By means of this special arrangement, the driver circuit is arranged very close to the optical elements to be controlled, as a result of which advantageous dynamics, in particular a high duty cycle, can be achieved.

### BRIEF DESCRIPTION OF THE DRAWINGS

Some embodiments are illustrated by way of example in the drawings and will be explained below, in which:

FIG. 1 shows a schematic illustration of an apparatus for stimulating an auditory nerve;

FIG. 2 shows a schematic illustration of an apparatus for stimulating an auditory nerve with a CMOS chip implemented as a layer stack;

FIG. 3 shows a lateral sectional view of a CMOS chip of an apparatus for stimulating an auditory nerve;

FIG. 4 shows a lateral sectional view of a CMOS chip of an apparatus for stimulating an auditory nerve with beam-shaping elements;

FIG. 5 shows a lateral sectional view of a CMOS chip of an apparatus for stimulating an auditory nerve with transparent regions;

FIG. 6 shows a lateral sectional view of a CMOS chip of an apparatus for stimulating an auditory nerve with optical elements emitting on two sides;

FIG. 7 shows a lateral sectional view of a CMOS chip of an apparatus for stimulating an auditory nerve with two sets of optical elements arranged on opposite sides; and

FIG. 8 shows a plan view of an apparatus for stimulating an auditory nerve.

### DETAILED DESCRIPTION OF THE INVENTION

In the following, examples of the present disclosure are described in detail and using the accompanying descriptions. In the following description, many details are described in order to provide a more thorough explanation of examples of the disclosure. However, it will be apparent to those skilled in the art that other examples can be implemented without these specific details. Features of the different examples described can be combined with one another, unless features of a corresponding combination are mutually exclusive or such a combination is expressly excluded.

It is to be noted that identical or similar elements or elements having the same functionality can be provided with identical or similar reference numerals or are denoted identically, wherein a repeated description of elements which are provided with identical or similar reference numerals or are denoted identically is typically omitted. Descriptions of elements which have identical or similar reference numerals or are denoted identically are interchangeable or mutually applicable.

To facilitate the description of the various embodiments, some of the figures have a Cartesian coordinate system x, y, z, wherein the x-y plane corresponds to, i.e. is parallel to, a main surface of a substrate (=a reference plane=x-y plane), wherein the direction perpendicular upwards with respect to the reference plane (x-y plane) corresponds to the “+z” direction, and wherein the direction perpendicular downwards with respect to the reference plane (x-y plane) corresponds to the “−z” direction. In the following description, the term “lateral” means a direction parallel to the x and/or y direction, i.e. parallel to the x-y plane, wherein the term “vertical” means a direction parallel to the z direction.

In addition, optical radiation is described by way of example using the example of light, for example radiation in a spectrum visible to humans. However, optical radiation in other wavelength ranges can also be used with the apparatus.

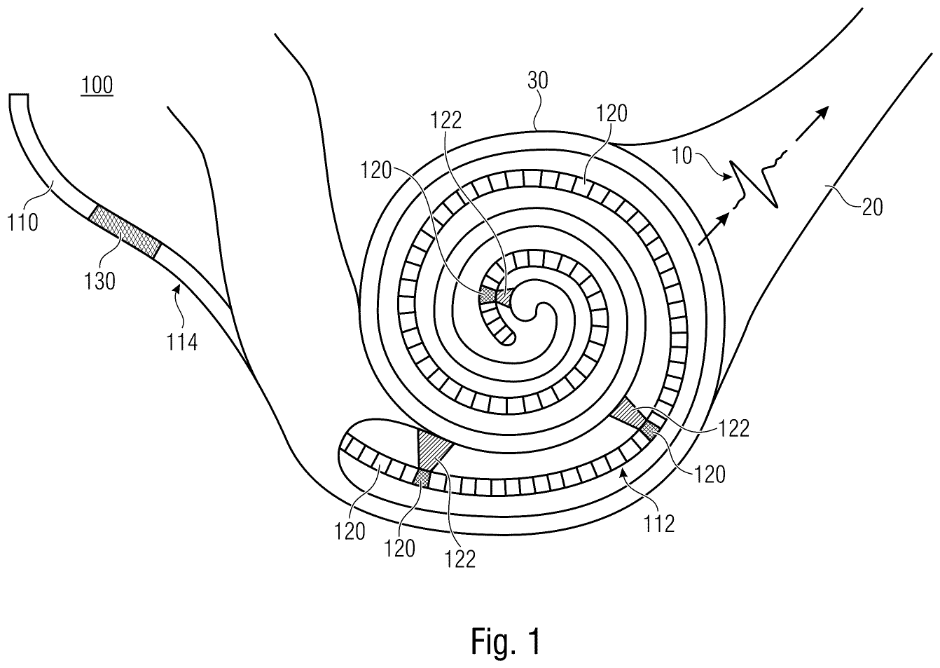

FIG. 1 schematically shows an apparatus 100 for stimulating 10 an auditory nerve 20. In FIG. 1, the apparatus is introduced at least partially into a cochlea 30. The cochlea 30 is illustrated in section in FIG. 1 in order to make the positioning of the apparatus 100 within the cochlea visible.

The apparatus 100 comprises a CMOS chip 110 in which a plurality of optical elements 120 and a driver circuit 130 are integrated.

The apparatus 100 can be divided into two portions, for example. A first portion 112 of the apparatus 100 can be introduced into the cochlea 30 and a second portion 114 of the apparatus 100 can be positioned outside the cochlea 30. The second portion 114 is located behind the ear of a user below the skin, for example. The first portion 112 and the second portion 114 form an inseparable unit, i.e. they share a substrate of the CMOS chip 110. The complete apparatus 100 is implantable.

The plurality of optical elements 120 are arranged in the first portion 112. The optical elements 120 of the plurality of optical elements 120 are configured to emit or radiate light 122 in order to stimulate 10 the auditory nerve 20. The optical elements 120 of the plurality of optical elements 120 are arranged linearly in the CMOS chip 110, for example. The plurality of optical elements 120 form a linear stimulation array in the CMOS chip 110, for example. In FIG. 1, for example, no distance between the optical elements 120 of the plurality of optical elements 120 is illustrated. However, it is clear that the optical elements 120 of the plurality of optical elements 120 can also be arranged to be spaced apart from one another within the CMOS chip 110.

The driver circuit 130 is, for example, arranged in the second portion 114 of the apparatus 100 in FIG. 1. However, it may be advantageous for the same to be also arranged in the first portion 112, in the vicinity of the plurality of optical elements 120. The driver circuit 130 is configured to control the plurality of optical elements 120. Individual optical elements 120 of the plurality of optical elements 120 can be controlled individually or several optical elements 120 of the plurality of optical elements 120 can be controlled simultaneously. FIG. 1 exemplarily shows the simultaneous driving of three optical elements 120.

Furthermore, it is possible for the apparatus 100 to have several driver circuits 130 and not just one. According to an embodiment, the apparatus 100 can, for example, have a plurality of driver circuits 130, wherein a driver circuit 130 of the plurality of driver circuits 130 is associated to each optical element 120 of the plurality of optical elements 120. The driver circuits 130 of the plurality of driver circuits 130 are configured to control the optical element 120 which is associated to the respective driver circuit 130. It is particularly advantageous for the plurality of driver circuits 130 to be arranged in the first portion 112 and not in the second portion 114. Thus, for example, an optical element 120 and a driver circuit 130 associated to this optical element 130 can be arranged in the immediate vicinity of one another within the CMOS chip.

According to an embodiment, a protective layer, e.g. a biocompatible protective layer, is arranged around the CMOS chip 110, i.e. the CMOS chip is encapsulated. The protective layer is transparent or semi-transparent at least in regions, e.g. at emission windows of the optical elements 120 or in the complete first portion 112 so that the light 122 of the plurality of optical elements 120 can be coupled out of the apparatus 100.

According to an embodiment, the apparatus 100 has a round cross-section. A diameter of the apparatus 100 is at most 120 μm, 110 μm, 100 μm or 90 μm. High flexibility of the apparatus 100 is achieved by this so that it can follow the shape of the cochlea 30. A diameter of at most 120 μm, 110 μm, 100 μm or 90 μm allows spiral bending of the apparatus 100 and facilitates introduction of the first portion 112 into the cochlea 30. Optionally, only the first portion 112 of the apparatus 110 has a diameter of at most 120 μm, 110 μm, 100 μm or 90 μm and the second portion 114 can also be realized with a larger diameter or other dimensions. However, it is particularly advantageous for the first portion and the second portion 114 to have the same dimensions, e.g. the same diameter or the same width and height.

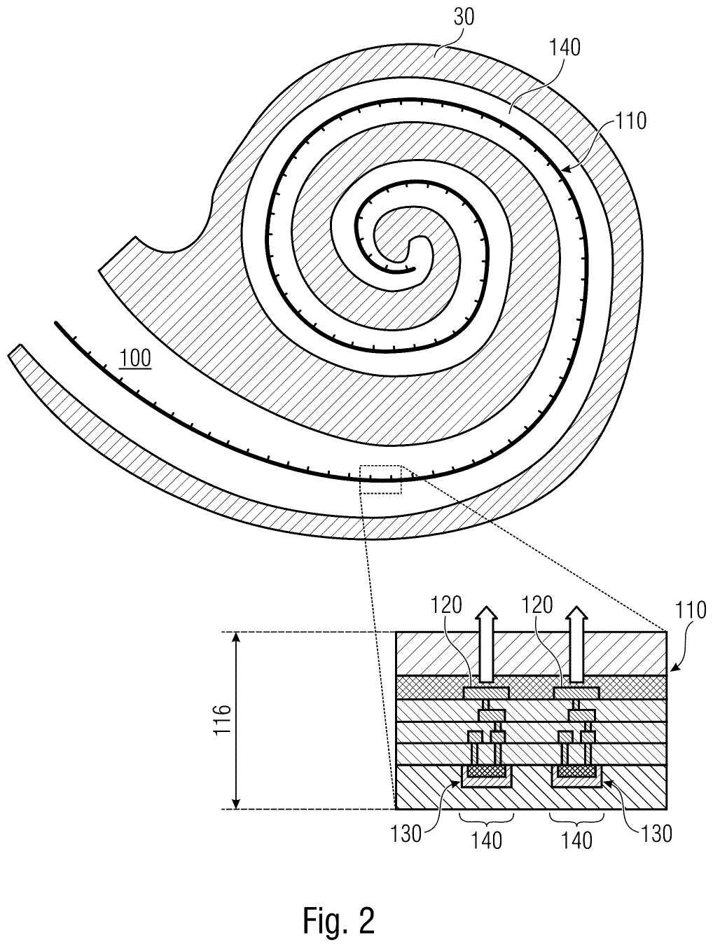

Further details of the apparatus 100 are illustrated below. The apparatus 100 can have features and/or functionalities as illustrated in connection with FIGS. 2 to 8.

An inventive arrangement, e.g., the apparatus 100, has only a single flexible CMOS chip 110, which in turn has a plurality of optically stimulating elements, e.g., the optical elements 120, and optionally further components, e.g., driver circuits 130. FIG. 2 illustrates this basic arrangement in a schematic cochlea 30. The advantage of this novel arrangement as a single CMOS chip 110 eliminates a plurality of construction problems of hybrid approaches.

Apart from CMOS, other production technologies could of course also be used.

FIG. 2 exemplarily shows how a plurality of optical elements 120 and a plurality of driver circuits 130 can be integrated in a layer stack of the CMOS chip 110. A respective driver circuit 130 and an optical element 120 associated to the driver circuit 130 form, for example, an electro-optically active element, i.e., an active stimulation element 140. A linear array of stimulation elements 140 is arranged in the CMOS chip 110, for example. The array of stimulation elements 140 forms, for example, a stimulation unit of the apparatus 100.

According to the embodiment in FIG. 2, the apparatus 100 can be implanted completely within the cochlea 30. Optionally, however, it is also possible for the apparatus 100, as described in connection with FIG. 1, to further have a second portion which can be positioned outside the cochlea 30.

The optically stimulating elements, i.e., the optical elements 120, are implemented, for example, as an integrable light source. These can be, for example, organic LEDs (light-emitting diodes), μLEDs or QDs (quantum dots).

The bendability of the substrate is generated, for example, by a thinning method. For this purpose, the silicon CMOS chip 110 is thinned, for example, to a thickness 116 of significantly below 100 μm. The thickness of the CMOS chip 110 is to be, for example, in a range of 10 μm-100 μm, 10 μm-70 μm, 10 μm-50 μm, 30 μm-100 μm, 30 μm-80 μm or 30 μm-60 μm. A possible lower limit can be 10 μm or 20 μm.

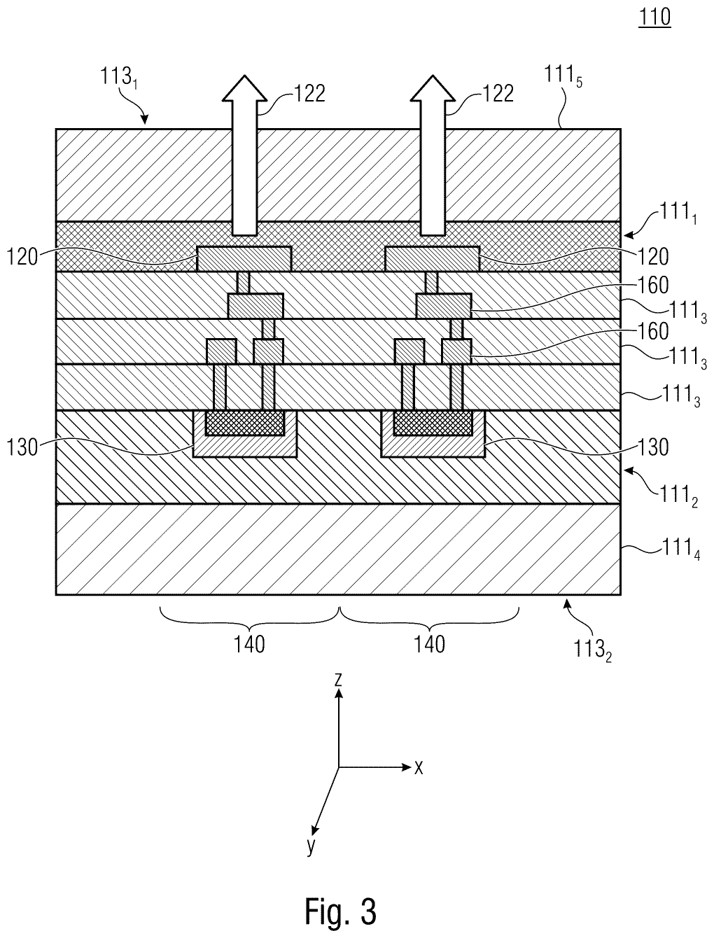

FIGS. 3 to 7 show exemplary detail views or enlarged schematic portions of the CMOS chip 110 of the apparatus 100 from FIG. 1 and/or of the apparatus 100 from FIG. 2.

In FIGS. 3 to 7, the CMOS chip is implemented as a layer stack. A schematic illustration of a cross-section through two electro-optically active elements 140 of the apparatus 100 is illustrated here. An electro-optically active element 140 comprises at least one optical element 120 and a driver circuit 130, which are arranged, for example, in different layers of the layer stack. The optical elements 120 of the plurality of optical elements 120 are arranged, for example, in a first layer 111₁ and the driver circuits 130 of the plurality of driver circuits 130 are arranged, for example, in a second layer 111₂ of the layer stack of the CMOS chip 110.

The CMOS chip 110 has, for example, a first main surface 113₁. The optical elements 120 of the plurality of optical elements 120 face the first main surface 113₁. The optical elements 120 of the plurality of optical elements 120 are configured, for example, to couple out light 122 from the CMOS chip 110 via the first main surface. Opposite the first main surface 113₁, the CMOS chip 110 has a second main surface 113₂, which is located, for example, in the reference plane (x-y plane). The first layer 111₁ is located above the second layer 111₂ in the layer stack direction, i.e. in the +z direction. The plurality of optical elements 120 are thus arranged within the layer stack between the first main surface 113₁ and the plurality of driver circuits 130.

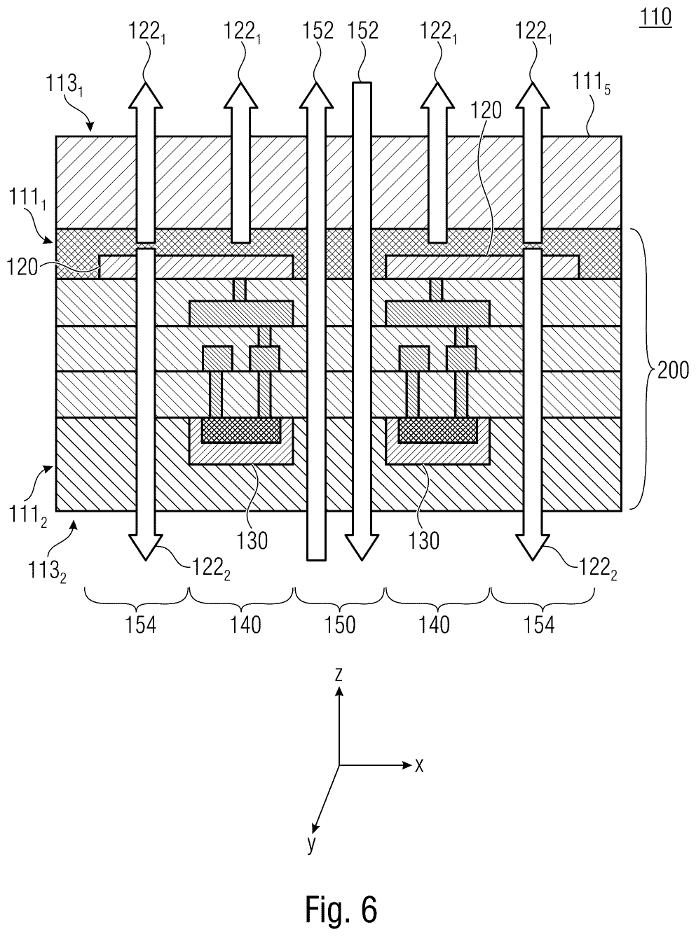

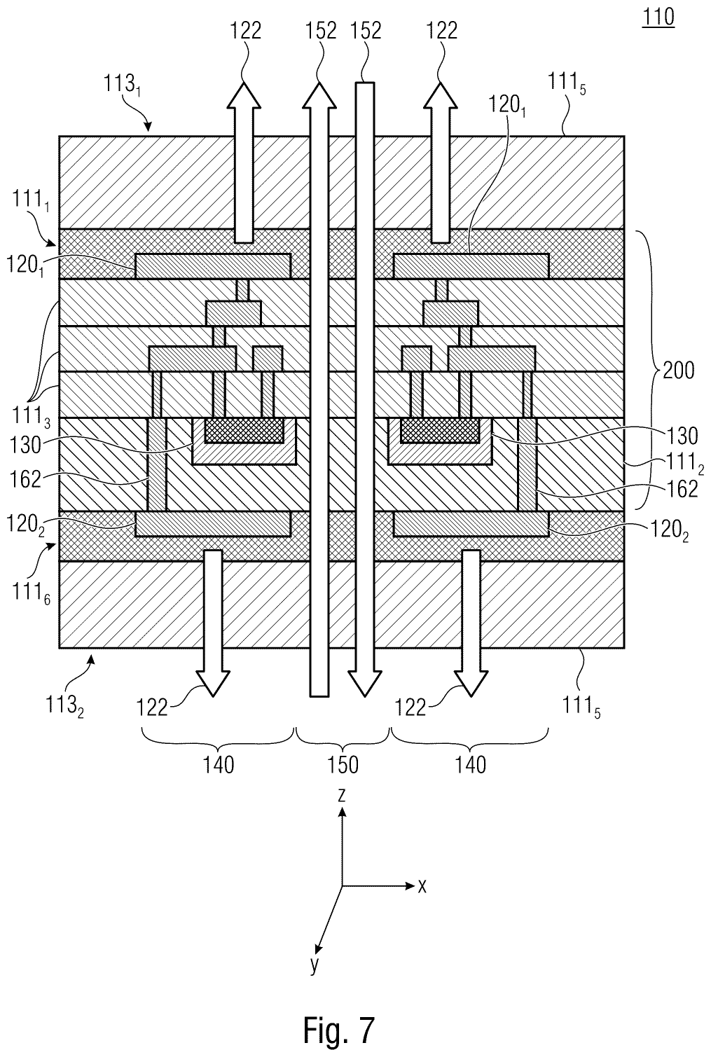

The optical elements 120 of the plurality of optical elements 120 and the driver circuits 130 of the plurality of driver circuits 130 can be arranged, for example, in their respective layer such that a driver circuit 130 and the associated optical element 120 are always arranged one above the other. In a plan view, for example, the driver circuit 130 and the associated optical element 120 overlap at least partially or completely. In FIGS. 3-5, the outer edges of the driver circuit 130 and of the associated optical element 120 are, for example, in alignment. However, this is not absolutely necessary, as can be seen, for example, in FIGS. 6 and 7. The axis of symmetry of the driver circuit 130 can thus be arranged to be offset to the axis of symmetry of the optical element.

Further layers 111₃ can be arranged between the first layer 111₁ and the second layer 111₂. These layers 111₃ are, for example, insulation layers in which electrical connections can be arranged. These layers 111₃ can have, for example, connection or wiring levels 160. These layers 111₃ can optionally have a transparent or semi-transparent material. The layers 111₃ represent, for example, transparent insulation layers. Only the connection or wiring levels 160 are made, for example, of an electrically conductive material which is, for example, non-transparent.

The first layer 111₁ corresponds, for example, to an optically active element, such as, for example, OLED (organic LED) or μLED (ultra LED), with a plurality of pixel electrodes, i.e., the optical elements 120.

The second layer 111₂ is, for example, a layer with electrically active components, e.g., CMOS transistors. A driver circuit 130 is formed, for example, from a plurality of electrically active components within the second layer 111₂.

A typical driver circuit consists of several active CMOS transistors and realizes, depending on the driver concept, a current or voltage source which supplies the optically active element with electrical energy. This current or voltage driving can additionally be combined with a modulation method. This can be, for example, a time-controlled pulse width modulation or another modulation with different signal shapes (e.g., sine, triangle, etc.).

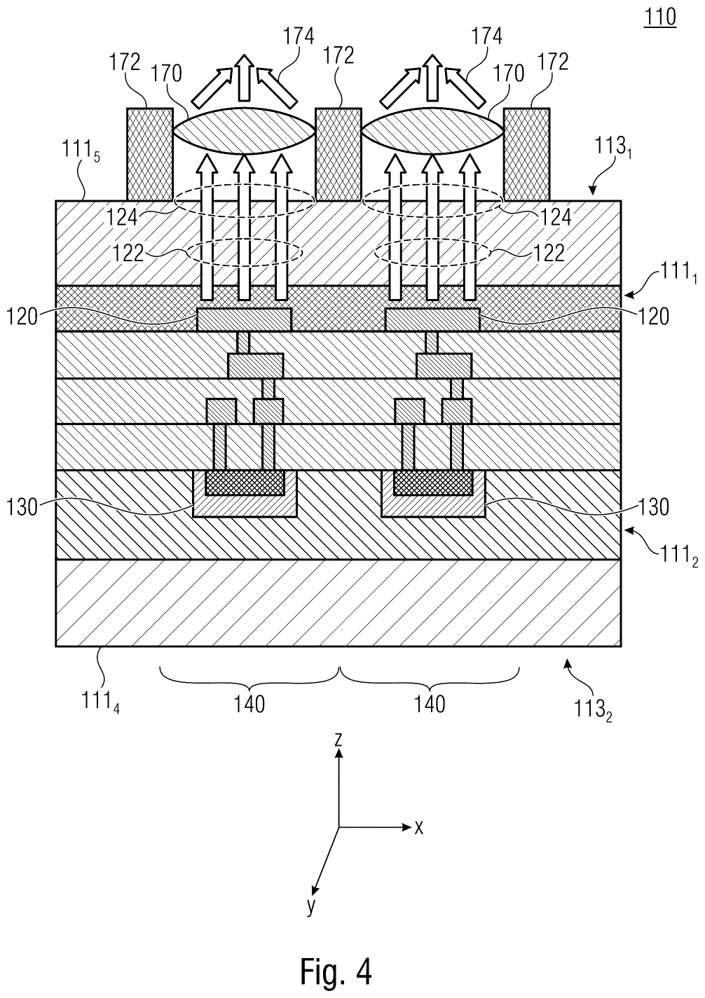

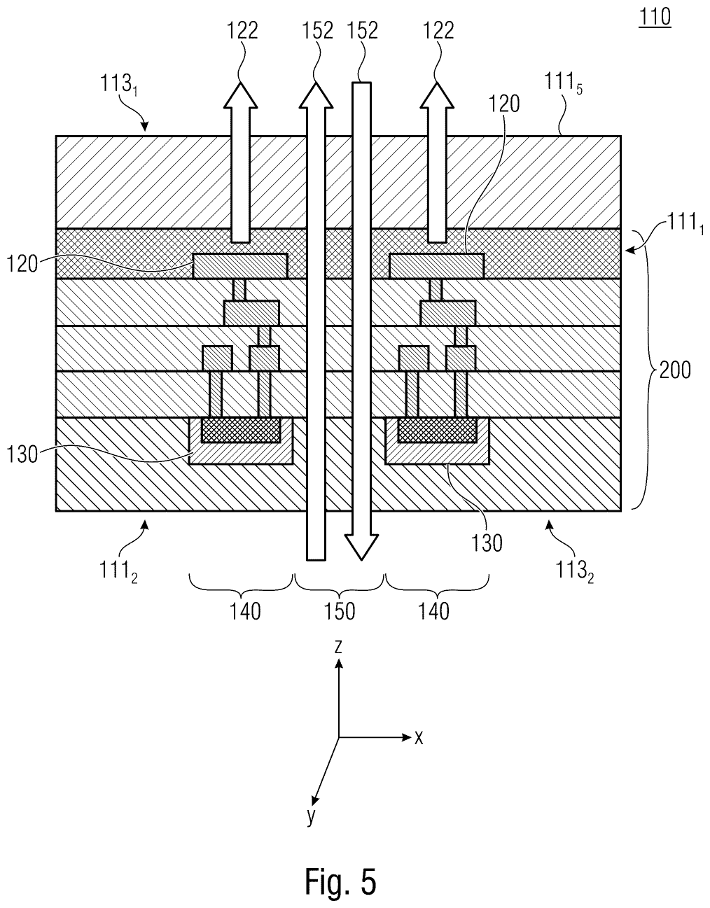

The second layer 111₂ can be arranged, for example, on a carrier substrate 1114, e.g., made of silicon material, e.g., a wafer substrate, see FIGS. 3 and 4. The carrier substrate 1114 can optionally also be removed, as can be seen, for example, in FIGS. 5 to 7. Consequently, the arrangement 200 becomes partially transparent 152. The CMOS chip 110 here comprises transparent regions 150 and non-transparent regions, e.g., the regions in which the electro-optically active elements 140 are arranged. Consequently, the light of the optical elements 120 can not only be coupled out of the first main surface 113₁ but can also be guided through the CMOS chip 110 and coupled out of the second main surface 113₂.

The CMOS chip 110 optionally has an encapsulation layer 111₅, e.g., a protective layer. The encapsulation layer 111₅ is made, for example, of a transparent or semi-transparent material. Optionally, the material of the encapsulation layer 111₅ is further biocompatible. The encapsulation layer 111₅ protects the apparatus 100 against external effects, on the one hand, and the user against harmful effects by the apparatus 100, on the other hand. The encapsulation layer 111₅ is arranged directly adjacent to the first layer 111₁ in the layer stack direction, i.e. in the +z direction, for example. An area of the encapsulation layer 111₅ facing away from the first layer 111₁ corresponds, for example, to the first main surface 113₁.

In order to realize good light power transmission to the auditory nerve, the arrangement can be supplemented by further beam-shaping elements 170, for example microlenses or fiber-optic components, as shown in FIG. 4, for example. The lateral optical scattering is reduced by such an arrangement and the frequency resolution achieved in the ear is thus improved. The beam-shaping elements 170 are arranged or fixed on the first main surface 113₁ of the CMOS chip 110. The optical elements 120 are configured, for example, to couple out light 122 within an emission region 124 via the first main surface 113₁. The beam-shaping elements 170 are arranged on the emission regions 124 and are configured to shape 174, for example bundle or focus, the light 122 of the respective optical element 120. For example, one of a plurality of beam-shaping elements 170 is arranged or fixed above each of the plurality of optical elements 120 in the layer stack direction. One, for example optically active, diaphragm 172, for example a light-impermeable element, is optionally arranged between two adjacent beam-shaping elements 170. The diaphragms 172 are configured, for example, to reduce optical over-coupling between the electro-optically active elements 140.

According to the embodiment in FIG. 6, semi-transparent pixel electrodes can be integrated into the CMOS chip 110 as the optical elements 120. Consequently, the light 122 is emitted away from the arrangement 200, see 122₁, and also through the arrangement 200, see 122₂. The light 122 can thus be coupled out both via the first main surface 113₁ and via the second main surface 113₂. Transparent regions 150 and non-transparent regions with upward light emission are formed, e.g., the regions in which the electro-optically active elements 140 are arranged, as well as partially transparent regions 154 with upward light emission, see 122₁, and downward, see 122₂.

The transparent regions 150 in the CMOS chip 110 have, for example, no optical elements 120, no connection or wiring levels 160 nor a driver circuit 130. The transparent regions are formed, for example, by the transparent material of the individual layers of the layer stack.

In the partially transparent regions 154, only the pixel electrode, i.e., the optical element 120, separates the layer stack into two transparent regions. The pixel electrode emits, for example, light 122 in two opposite directions, e.g., in the direction of the first main surface 113₁ and in the direction of the second main surface 113₂. The two transparent regions separated from each other by the pixel electrode are formed, for example, by the transparent material of the individual layers of the layer stack. Partially transparent regions 154 are formed in the CMOS chip 110, for example, in that, in a plan view, the pixel electrode extends over a larger area than a driver circuit 130 and/or connection or wiring levels 160 at least partially overlapping with the pixel electrode in the plan view. The overlap region is regarded as a non-transparent region, and the region extending beyond and defined by the pixel electrode is regarded as a partially transparent region 154.

FIG. 7 shows an alternative embodiment with an additional pixel electrode on the lower side of the arrangement 200. For this purpose, a via 162, for example, is introduced into the layer with the active components, i.e., into the second layer 111₂, in order to electrically connect the pixel electrode. In this case, the plurality of optical elements 120 has, for example, a first set of optical elements, see 1201, and a second set of optical elements, see 120₂. The optical elements 1201 of the first set of optical elements 120 are arranged, for example, in the first layer 111₁ and the optical elements 120₂ of the second set of optical elements 120 are arranged, for example, in a third layer 111₆ of the layer stack. The second layer 111₂ and optionally further layers 111₃ are arranged between the first layer 111₁ and the third layer 111₆. According to an embodiment, two opposite optical elements 120 always share a driver circuit 130 arranged therebetween. According to FIG. 7, two optical elements 120 and a driver circuit 130 can thus form an electro-optically active element 140 within the CMOS chip 110.

According to an embodiment, all components of an electro-optically active element 140 are aligned with one another within the layer stack of the CMOS chip 110 such that they are arranged one above the other and, for example, occupy as little area as possible as seen in the plan view.

Like the first layer 111₁, the third layer 111₆ can correspond to an optically active element, such as, for example, OLED (organic LED) or μLED (ultra LED), with a plurality of pixel electrodes, i.e., the optical elements 120.

Optionally, a further encapsulation layer 111₅ is arranged below and directly adjacent to the third layer 111₆ in the layer stack direction, i.e. in the +z direction. The CMOS chip 110 is thus optionally encapsulated. The further encapsulation layer 111₅ is made, for example, of the same material as already described above in connection with the encapsulation layer 111₅ on the first layer 111₁ and can also have the same properties. An area of the encapsulation layer 111₅ facing away from the third layer 111₆ corresponds, for example, to the second main surface 113₂.

As can be seen in FIG. 7, transparent regions 150 and non-transparent regions are formed, see 140. In the region of the non-transparent regions, light 122 can be emitted from both sides in this setup since, for example, the optical elements 120 of the first set of optical elements 1201 face the first main surface 113₁ and the optical elements 120₂ of the second set of optical elements 120 face the second main surface 113₂.

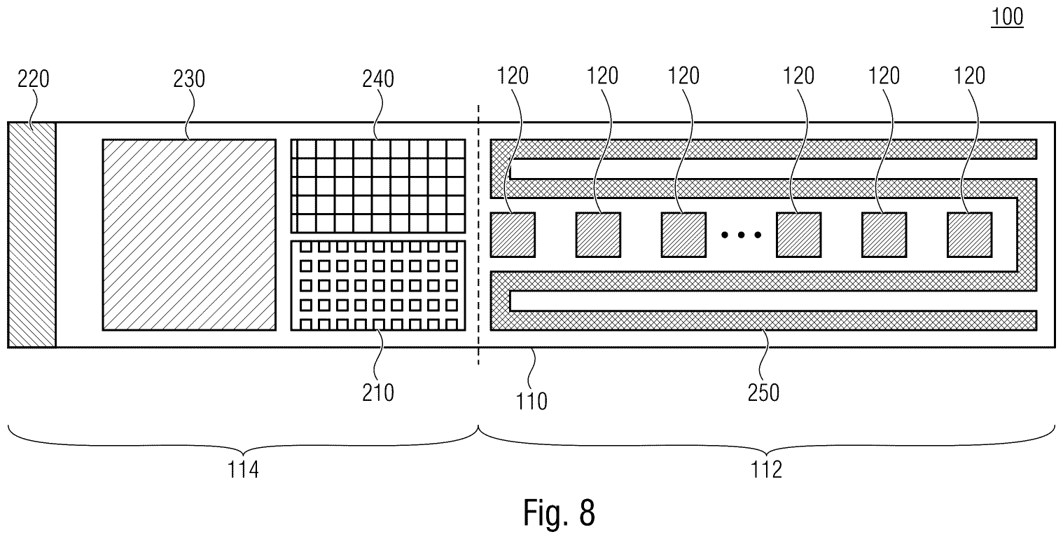

Using a CMOS chip 110 allows the integration of further components, as can be seen in the schematic plan view of the apparatus 100 in FIG. 8. In addition to the driver circuit 130 (arranged below the optical elements 120) for the optical elements 120, further components can also be integrated, such as, for example, a speech processor/audio processor/sound processor 210, a data and/or energy transmission unit 220, e.g. a wireless interface for power supply and external data transmission or a contact series, e.g. for a direct connection, up to the integrated microphone 230. Alternatively or additionally, a wireless RF interface 240 and/or a fully integrated RF antenna 250 (radio frequency antenna) can be integrated optionally. The RF antenna is used, for example, for communication with the external unit or further external devices. The positioning does not have to be in the region of the stimulation units. Possible further positions with respect to the surroundings would probably even be more favorable for positioning outside. Due to the greater length of the stimulation unit, the positioning of the antenna next to these would possibly be advantageous (depending on the RF wavelengths used).

Such a “fully integrated” system could be implanted while encapsulated in the ear. Any connections to the outside and potential infections associated therewith could be completely avoided, for example if the wireless interface is implemented and not the series of contacts.

The apparatus 100 can have two portions, wherein a first portion 112 can be introduced into the cochlea and a second portion 114 can be positioned outside the cochlea.

The energy supply of the system, i.e. the apparatus 100, can be realized by different variations:
Autonomous energy harvesting from cell energy, chemical energy or motion energy, generally “bio-energy”
Externally supplied by energy coupled directly into the chip or the package, for example via:
   Electrical field
   Magnetic field
   Light, advantageously infrared
   Mechanically

The present invention describes a fully integrable cochlear implant, i.e. the apparatus 100. The core of the invention is the very high integration density of elements for stimulating the auditory nerve, i.e. the optical elements 120, a corresponding signal processing, e.g. the speech processor/audio processor/sound processor 210, and a wireless interface 220 for data transmission in a single, encapsulated chip, with simultaneous implantability in the auditory canal.

General advantages:
Complex method is simplified drastically
Standard methods of semiconductor technology are used
Single substrate for all components, no SCT required
Integrated approach enables more optical elements and thus frequency resolution
Light sources can be positioned at a very small distance from one another, i.e., higher density of the light sources per mm possible than in the case of μLEDs
Light can be coupled out on both sides of the CMOS chip.

Further embodiments of the apparatus 100 described above are explained below, which can be included individually or in combination in the embodiments described above.

An embodiment relates to an optoelectronic hearing aid, i.e., the apparatus 100, which enables direct stimulation of the auditory nerve in the event of damage to the inner ear. The apparatus 100 has an active, optically stimulating component, see 140 in FIGS. 3 to 7, which is integrated directly on a CMOS chip 110. Furthermore, the apparatus 100 has further integrated electronics for preprocessing and driving the active optical components, see 140. The entire system, i.e., the apparatus 100, has high flexibility, i.e., the thickness of the CMOS chip 110 is less than 100 μm.

In addition to the active optical components, see 140, further passive optical components can be integrated into the CMOS chip 110. Possible embodiments for these passive optical components are optical filters which change the spectral behavior or the polarization of the produced light 122. Examples of these are absorption filters, dielectric mirrors, metal grid filters, plasmonic filters, etc.

The active, optically-stimulating elements are supplemented partially or completely by further electrical elements.

Parts of the integrated electronics are not flexible, for example.

The integrated electronics have, for example, acoustic data preprocessing, see 210 in FIG. 8, a wireless interface (for example inductive or optical) for data transmission and/or energy supply, see 220 in FIG. 8, and/or a microphone, see 230 in FIG. 8.

According to an embodiment, the integrated electronics are supplemented by an electrical energy store integrated in the CMOS chip 110.

The entire system can additionally have an encapsulation layer. The encapsulation layer 111₅ discussed in FIGS. 3 to 7 can enclose or encapsulate, for example, the entire CMOS chip 110.

The flexible CMOS chip 110 is partially transparent.

A technology other than CMOS is used, for example, as an active driver circuit, such as, for example, TFTs (e.g., a-Si, LTPS, IGZO, organic field effect transistors), III-V semiconductors, SiC, etc.

The apparatus 100 can have an additional element for bio-energy harvesting (“bio-energy harvester”) for an autonomous power supply of the entire system.

The energy used for the apparatus 100 is introduced directly into the chip or the package, for example, from outside.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

### REFERENCES

[1] Adam Kissiah, Implantable Electronic Hearing Aid, Patent US000004063048, 1978
[2] Dieter et al., “μLED-based optical cochlear implants for spectrally selective activation of the auditory nerve”, EMBO Molecular Medicine, 2020
[3] Keppeler et al., “Multichannel optogenetic stimulation of the auditory pathway using microfabricated LED cochlear implants in rodents”, SCIENCE TRANSLATIONAL MEDICINE, 2020

1. An apparatus for stimulating an auditory nerve, comprising:
a chip;
a plurality of optical elements for stimulating the auditory nerve; and
at least one driver circuit for controlling the plurality of optical elements;
wherein the plurality of optical elements and the at least one driver circuit are integrated monolithically in the chip; and
wherein the chip is implemented as a CMOS chip.

2. The apparatus according to claim 1, wherein the chip comprises a TFT, in particular a-Si, LTPS or IGZO with or without organic field effect transistor, or a III-V semiconductor material or SiC material.

3. The apparatus according to claim 1, wherein the apparatus, for each of the plurality of optical elements, comprises a driver circuit, for controlling the respective optical element, which is integrated in the chip.

4. The apparatus according to claim 1, wherein one or more of the following components are also integrated in the chip:
a speech processor,
a wireless interface,
a microphone, and
an energy supply for providing energy acquired from electromagnetic energy, optical energy, cell energy, chemical energy, thermal energy or motion energy.

5. The apparatus according to claim 4,
wherein the chip comprises a first portion, which is configured to be introduced into a cochlea, and a second portion, which is configured to be arranged outside the cochlea, and
wherein the plurality of optical elements and the at least one driver circuit are integrated in the first portion of the chip, and
wherein the one or more components are integrated in the second portion of the chip.

6. The apparatus according to claim 1, comprising an RF antenna, which is integrated in the chip.

7. The apparatus according to claim 1, wherein the chip comprises a thickness of at most 100 μm.

8. The apparatus according to claim 1, comprising at least one beam-shaping element,
wherein the chip comprises a first main surface,
wherein an optical element of the plurality of optical elements is configured to couple out light within an emission region via the first main surface,
wherein the at least one beam-shaping element is arranged in the emission region on the first main surface.

9. The apparatus according to claim 1, wherein the chip comprises a first main surface and a second main surface opposite the first main surface, and
wherein the chip comprises a transparent or semi-transparent region for coupling out light of the plurality of optical elements via the first main surface and via the second main surface.

10. The apparatus according to claim 1, wherein the optical elements of the plurality of optical elements are configured to emit light in two opposite directions.

11. The apparatus according to claim 1, wherein the chip is configured as a layer stack and the plurality of optical elements are arranged in a first layer of the layer stack and the at least one driver circuit is arranged in a second layer of the layer stack.

12. The apparatus according to claim 1, wherein the chip is configured as a layer stack,
wherein the plurality of optical elements comprise a first set of optical elements and a second set of optical elements, and
wherein the first set of optical elements is arranged in a first layer of the layer stack, facing a first main surface of the chip, and wherein the second set of optical elements is arranged in a second layer of the layer stack, facing a second main surface of the chip, wherein the second main surface corresponds to an area of the chip opposite the first main surface.

13. The apparatus according to claim 12, wherein the at least one driver circuit is arranged in a third layer of the layer stack, and wherein the third layer is arranged between the first layer and the second layer.

14. The apparatus according to claim 1, wherein, in a plan view, the at least one driver circuit overlaps at least partially with at least one of the plurality of optical elements, or wherein, in the plan view, the at least one driver circuit is arranged between two adjacently arranged optical elements of the plurality of optical elements.
